(19) United States
(12) Patent Application Publication
(10) Pub. No.: US 2024/0164916 A1
(43) Pub. Date: May 23, 2024

(54) ADJUSTABLE OVERLAYING LINER-PROSTHESIS INTERFACE AND CORRESPONDING METHOD

(71) Applicant: ETHNOCARE INC., Québec (CA)

(72) Inventors: Marc-Antoine MALOUIN-LIZOTTE, Québec (CA); Louis-Philippe GARNEAU, Québec (CA); Vincent BRETON, Québec (CA)

(73) Assignee: ETHNOCARE INC., Québec (CA)

(21) Appl. No.: 18/280,234

(22) PCT Filed: Mar. 3, 2022
(86) PCT No.: PCT/CA2022/050305

§ 371 Date: Sep. 3, 2023

Related U.S. Application Data
(60) Provisional application No. 63/155,930, filed on Mar. 3, 2021.

(51) Int. Cl. A61F 2/78; A61F 2/50; A61F 2/74; A61F 2/80

(52) U.S. Cl. CPC A61F 2/7843; A61F 2/748; A61F 2/80; A61F 2002/5032; A61F 2002/7837

(57) ABSTRACT

An adjustable overlaying interface for adjusting a prosthesis to a liner worn over a residual limb of a user comprises a sleeve, an inflatable bladder and a fluid-providing device. The variations in volume of the residual limb is compensated by using the fluid-providing device to inflate and/or deflate the inflatable bladder. A kit for adjusting the prosthesis to the residual limb comprises a liner and the adjustable overlaying interface. A method for mounting the prosthesis to the residual limb of a user comprises installing the adjustable overlaying interface over a liner donned over the residual limb of the user, at least partially inserting the residual limb wearing the liner and the adjustable overlaying interface within a socket of the prosthesis and inflating or deflating the inflatable bladder of the adjustable overlay interface to compensate variations in volume of the residual limb.

### TECHNICAL FIELD

The technical field generally relates to limb-prosthesis interfaces, and more particularly to adjustable overlaying liner-prosthesis interfaces, and to corresponding methods.

### BACKGROUND

Prosthetic liners are commonly used to act as a barrier or interface between an amputated or residual limb (or stump) and a socket of a prosthesis, for the purpose of increasing comfort and maintaining a consistent fit of the prosthesis onto the residual limb. Nevertheless, most liners do not account for the variation in volume, decrease in blood flow in the limb, increased temperature of the limb, and/or any pain and injury commonly related to the prolonged use of a prosthesis. Moreover, some users find known liners to be uncomfortable due to a poor pressure distribution resulting from the limb-liner-socket interface. When using a prosthetic socket, normal and shear forces are generated upon the skin of the residual limb. Many known liners fail to distribute pressure evenly about the residual limb, causing areas of the limb to be subject to pressure greater than others. The concentrated pressure areas can cause discomfort, pain, and pressure marks. To overcome these concerns, the prosthesis would normally need to be removed multiple times a day or multiple layers of socks or liners would need to be applied. Nevertheless, this only temporarily treats the discomfort and is not an effective solution.

U.S. Pat. No. 10,806,606 discloses a liner system for reducing motion between a socket of a prosthesis and a limb inserted therein. The liner system teaches using shims placed around the limb to compensate for variation in limb volume. One drawback of such liner system is that it forces the user to purchase a complete new system including a new liner to benefit from the advantages promised by such liner system.

In view of the above, there is a need for a limb-prosthesis interface which would be able to overcome or at least minimize some of the above-discussed prior art concerns.

### SUMMARY

It is an object of the present invention to provide an adjustable overlaying interface for adjusting a prosthesis to a liner worn over a residual limb of a user that overcomes or mitigates one or more disadvantages of known such adjusting systems, or at least provides a useful alternative. The invention provides the advantage of being compatible with standard liners and prosthesis that a user likely already owns. Moreover, the invention provides the advantage of being adapted to locally compensate where the limb volume varies rather than generally all around the limb.

In accordance with an embodiment of the present invention, there is provided an adjustable overlaying interface for adjusting a prosthesis to a liner worn over a residual limb of a user. The adjustable overlaying interface comprises a sleeve, an inflatable bladder and a fluid-providing device. The sleeve is pullable on an outer surface of the liner, thereby being adapted to be worn over the liner and at least partially inside a socket of the prosthesis. The inflatable bladder is attached to or integrally formed with the sleeve. The fluid-providing device is fluidly connected to the inflatable bladder and is operative to inflate the inflatable bladder with a fluid. The variations in volume of the residual limb may therefore be compensated by using the fluid-providing device to inflate and/or deflate the inflatable bladder.

Optionally, the adjustable overlaying interface may further comprise a valve fluidly connected to the inflatable bladder in order to selectively let the fluid out of the inflatable bladder.

Optionally, the sleeve is at least partially made of a stretchable material so as to snugly fit over the liner. At least a portion of the sleeve may have a stretchability of at least 10% and may have a tensile module of less than substantially 100 kPa. The sleeve material may at least partially be made of an antibacterial material and may at least partially made of a fabric. Optionally, the sleeve may at least be partially made of a material selected from the list consisting of: nylon, polyester, elastane, aramid fibers, cotton, wool, silk, thermoplastic polyurethane, polyurethane and polyvinyl chloride or a combination thereof.

Optionally, at least a portion of an inner surface of the sleeve may have a sleeve friction coefficient against human skin of less than substantially 1. The inner surface of the sleeve may have a first zone and a second zone, in which case the first zone represents at least 60% of a total inner surface area of the sleeve and may have a friction coefficient against human skin of less than 0.5. The second zone may have a friction coefficient against human skin greater than the friction coefficient of the first zone and less than 1. The second zone may be arranged at at least one of distal and proximal end portions of the sleeve.

Optionally, the inflatable bladder may be permanently attached to the sleeve. The inflatable bladder may be secured to an outer surface of the sleeve whereas at least a portion of the outer surface of the sleeve is adapted to face an inner surface of the socket. The inflatable bladder may extend over less than 270 degrees of a circumference of the sleeve or even less than 180 degrees of the circumference of the sleeve, but more than 90 degrees of the circumference of the sleeve.

The inflatable bladder comprises two walls sealed together on a periphery thereof. Optionally, the two walls are further attached together locally and within their periphery so as to define relief zones where the inflatable bladder is uninflatable and a plurality of inflatable cells where the inflatable bladder can be inflated. The inflatable cells create a visible pattern on at least a portion of an outer surface of the adjustable overlaying interface, the outer surface being adapted to face at least a portion of the inner surface of the socket. The pattern is indicative of an adjustable zone to be aligned over a predetermined muscular portion of the residual limb of the user that is prone to varying in volume. At least one of the plurality of inflatable cells may be smaller than a circle of 60 mm of diameter. The pattern may extend along a longitudinal axis of the sleeve.

Optionally, the inflatable bladder may be made, at least partially, of a stretchable material. The material also be the same material as used for the sleeve. The inflatable bladder may use a film made of a bladder material impervious to the fluid. This bladder material may be selected from the list consisting of: thermoplastic polyurethane, thermoplastic elastomer, polyurethane, polyvinyl chloride or a combination thereof.

Optionally, the thickness of the adjustable overlaying interface may be less than 3 mm, or even less than 2 mm, along substantially an entirety of a periphery thereof when the inflatable bladder is fully deflated. It may even be less than that, for example, less than 1.5 mm.

Optionally, the fluid-providing device may be adapted to pressurize and pump a compressible fluid. Both the fluid-providing device and the valve may be located proximate a longitudinal extremity of the sleeve so as to be outside the socket when in use. The fluid-providing device may be longitudinally spaced apart from the inflatable bladder. The fluid-providing device may be one of a pump and a cartridge of pressurized compressible fluid such as a CO₂ cartridge.

In accordance with another embodiment of the present invention, there is provided a prosthetic liner-recovering sheath for use with a prosthetic socket and an associated liner. The prosthetic liner-recovering sheath comprises a sheath body and a thickness-adjusting system. The sheath body is substantially tubular along at least a longitudinal portion thereof and has a liner-superposing inner surface at least partially defining a liner-receiving cavity. The thickness-adjusting system is secured to or is integrally formed with the sheath body and comprises at least one variable-volume pad arranged to be pressurized so as to adjust a thickness of the sheath along at least a portion of a periphery thereof.

Optionally, the thickness-adjusting system further comprises a fluid-providing device in fluid communication with the at least one variable-volume pad to respectively inflate and deflate the at least one variable-volume pad. The thickness-adjusting system may further comprises a valve which is fluidly connected to the at least one variable-volume pad in order to selectively let the fluid out of the at least one variable-volume pad.

Optionally, the sheath body is at least partially made of a stretchable material so as to snugly fit over the liner. At least a portion of the sheath body may have a stretchability of at least 10% and may have a tensile module of less than substantially 100 kPa. The sheath may at least partially be made of an antibacterial material and may at least partially made of a fabric. Optionally, the sheath body may at least be partially made of a material selected from the list consisting of: nylon, polyester, elastane, aramid fibers, cotton, wool, silk, thermoplastic polyurethane, polyurethane and polyvinyl chloride.

Optionally, at least a portion of an inner surface of the sheath body may have a sheath body friction coefficient against human skin of less than substantially 1. The inner surface of the sheath body may have a first zone and a second zone, in which case the first zone represents at least 60% of a total inner surface area of the sheath body and may have a friction coefficient against human skin of less than 0.5. The second zone may have a friction coefficient against human skin greater than the friction coefficient of the first zone and less than 1. The second zone may be arranged at at least one of distal and proximal end portions of the sheath body.

Optionally, the variable-volume pad may be permanently attached to the sheath body. The variable-volume pad may be secured to an outer surface of the sheath body whereas at least a portion of the outer surface of the sheath body is adapted to face the inner surface of the socket. The variable-volume pad may extend over less than 270 degrees of a circumference of the sheath body or even less than 180 degrees of the circumference of the sheath body, but more than 90 degrees of the circumference of the sheath body.

The variable-volume pad comprises two walls sealed together on a periphery thereof. Optionally, the two walls are further attached together locally and within their periphery so as to define relief zones where the variable-volume pad is uninflatable and a plurality of inflatable cells where the variable-volume pad can be inflated. The inflatable cells create a visible pattern on at least a portion of an outer surface of the adjustable overlaying interface, the outer surface being adapted to face at least a portion of the inner surface of the socket. The pattern is indicative of an adjustable zone to be aligned over a predetermined muscular portion of the residual limb of the user that is prone to varying in volume. At least one of the plurality of inflatable cells may be smaller than a circle of 60 mm of diameter. The pattern may extend along a longitudinal axis of the sheath body.

Optionally, the variable-volume pad may at least partially be made of a stretchable material. The material also be the same material as used for the sheath body. The variable-volume pad may use a film made of a bladder material impervious to the fluid. This bladder material may be selected from the list consisting of: thermoplastic polyurethane, thermoplastic elastomer, polyurethane, polyvinyl chloride.

Optionally, the thickness of the adjustable overlaying interface may be less than 3 mm, or even less than 2 mm, along substantially an entirety of a periphery thereof when the variable-volume pad is fully deflated. It may even be less than that, for example, less than 1.5 mm.

Optionally, the fluid-providing device may be adapted to pressurize a compressible fluid. Both the fluid-providing device and the valve may be located proximate a longitudinal extremity of the sheath body so as to be outside the socket when in use. The fluid-providing device may be longitudinally spaced apart from the variable-volume pad. The fluid-providing device may be one of a pump and a cartridge of pressurized compressible fluid such as a CO₂ cartridge.

In accordance with another embodiment of the present invention, there is provided a prosthetic liner-recovering sheath for use with a prosthetic socket and an associated liner. The prosthetic liner-recovering sheath comprises a sheath body and a thickness-adjusting system. The sheath body is substantially tubular along at least a longitudinal portion thereof and has a liner-superposing inner surface at least partially defining a liner-receiving cavity. The thickness-adjusting system is secured to or is integrally formed with the sheath body and comprising at least one variable-volume pad arranged to be pressurized so as to adjust a thickness of the sheath along at least a portion of a periphery thereof.

In accordance with another embodiment of the present invention, there is provided a kit for adjusting a prosthesis to a residual limb of a user. The kit comprises an inner member and an adjustable outer sheath. The inner member has a limb-facing surface at least partially delimiting a limb-receiving cavity and being superposable onto the residual limb of the user. The inner member also has an opposed outer surface. The adjustable outer sheath comprises a sheath body and a thickness-adjusting system. The sheath body overlays at least partially the outer surface of the inner member. The thickness-adjusting system is secured to or is formed integral with the sheath body. The thickness-adjusting system comprises at least one variable-volume bladder arranged to be pressurised so as to adjust a thickness of the adjustable outer sheath along at least a portion of a periphery thereof.

In accordance with another embodiment of the present invention, there is provided a kit for adjusting a prosthesis to a residual limb of a user. The kit comprises 1) an inner member having a limb-facing surface at least partially delimiting a limb-receiving cavity and being superposable onto the residual limb of the user and an opposed outer surface; and 2) the prosthetic liner-recovering sheath as previously described and overlaying at least partially the outer surface of the inner member.

In accordance with another embodiment of the present invention, there is provided a method for mounting a prosthesis to a residual limb of a user. The method comprises:
a) providing the adjustable overlay interface as described here above;
b) installing the adjustable overlaying interface over a liner donned over the residual limb of the user;
c) at least partially inserting the residual limb wearing the liner and the adjustable overlaying interface within a socket of the prosthesis; and
d) at least one of inflating and deflating the inflatable bladder of the adjustable overlay interface to compensate variations in volume of the residual limb.

Optionally, the action of b) installing the adjustable overlaying interface may comprise pulling the sleeve of the adjustable overlaying interface over the residual limb. The method may further comprise the action of e) aligning the inflatable bladder of the adjustable overlaying interface over a predetermined muscular portion of the residual limb that is prone to varying in volume. The method may also comprise the action of f) inflating the inflatable bladder via the fluid-providing device. The inflatable bladder may be inflated to a predetermined inner pressure. The method may further comprise deflating the inflatable bladder to the predetermined pressure using the valve.

In accordance with another embodiment of the present invention, there is provided an adjustable residual limb-prosthesis interface, comprising: a residual limb-engaging sleeve engageable with a residual limb of a user; and a thickness-adjusting system comprising: at least one inflatable air bladder being part of or being mounted to the residual limb-engaging sleeve; and an air pump and a valve both being in fluid communication with said at least one inflatable air bladder to respectively inflate and deflate said at least one inflatable air bladder so as to adjust a thickness of the adjustable residual limb-prosthesis interface.

In accordance with another embodiment of the present invention, there is provided a prosthetic liner device for forming an interface between a residual limb and a prosthesis socket, comprising: a) a liner adapted for at least partially engaging said residual limb; b) an air pump system affixed to or being part of said liner and comprising a pump, at least one inflatable air bladder, and a valve; wherein dimensions of a gap between an outer surface of said liner and an inner surface of the prosthesis socket are adjusted by inflating and/or deflating said at least one inflatable air bladder.

In accordance with another embodiment of the present invention, there is provided a kit comprising the prosthetic liner device described herein, and at least one of a prosthesis and a prosthesis socket.

In accordance with another embodiment of the present invention, there is provided a limb prosthesis interface cushioning device for forming at least partially an interface between a residual limb and a prosthesis, the cushioning device defining a limb-receiving cavity and comprising at least one bone-contacting area at least partially made of silicone and delimiting at least partially the limb-receiving cavity.

In accordance with another embodiment of the present invention, there is provided a method for reducing discomfort, pain, heat accumulation, and/or risk of injury caused by a prolonged used of a prosthesis equipping a residual limb, said method comprising: providing a prosthetic liner device described herein; engaging the liner with the residual limb; engaging a prosthesis socket of said prosthesis with the prosthetic liner device; and actuating at least one of the pump and the valve to inflate and/or deflate said at least one inflatable air bladder to modify dimensions of a gap defined between an outer surface of the liner and an inner surface of a prosthesis socket.

In accordance with another embodiment of the present invention, there is provided a method for adjusting a cooperation between a residual limb and a prosthesis, comprising: providing an adjustable residual limb-prosthesis interface described herein; engaging the residual limb-engaging sleeve with the residual limb; engaging a prosthesis socket of said prosthesis with the adjustable residual limb-prosthesis interface; if a thickness of the residual limb-engaging sleeve is smaller than a space between the residual limb and an inner surface of the prosthesis socket, actuating the air pump to inflate said at least one inflatable air bladder; and if a pressure within the at least one inflatable air bladder is greater than a predetermined pressure, actuating the valve to deflate said at least one inflatable air bladder.

In accordance with another embodiment of the present invention, there is provided a method for adjusting a cooperation between a residual limb and a prosthesis, comprising: providing an adjustable residual limb-prosthesis interface described herein; engaging the residual limb-engaging sleeve with an existing prosthetic liner or sock that is applied on the residual limb; engaging a prosthesis socket of said prosthesis with the adjustable residual limb-prosthesis interface; if a thickness of the residual limb-engaging sleeve is smaller than a space between the residual limb and an inner surface of the prosthesis socket, actuating the air pump to inflate said at least one inflatable air bladder; and if a pressure within the at least one inflatable air bladder is greater than a predetermined pressure, actuating the valve to deflate said at least one inflatable air bladder.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a front view of a user wearing a prosthesis with an adjustable overlaying interface in accordance with an embodiment of the present invention;

FIG. 2 is an exploded front view of the arrangement of FIG. 1;

FIG. 3 is an axonometric view from the front and top showing the adjustable overlaying interface of FIG. 1;

FIG. 4A is a cross-sectional view of the adjustable overlaying interface as shown in FIG. 3 taken along cross-section lines 4A-4A thereof;

FIG. 4B is the same cross-sectional view of the adjustable overlaying interface as in FIG. 4A, but with the addition of a cross-section of an installed socket of the prosthesis in order to illustrate a space there in between and showing an inflatable bladder in an under-inflated state;

FIG. 4C is the same cross-sectional view of the adjustable overlaying interface as in FIG. 4B, but depicting the inflatable bladder in an inflated state;

FIGS. 5A-5G are examples of various possible designs of thickness-adjusting systems for use in the adjustable overlaying interface of FIG. 1;

FIG. 6 is a side view of an adjustable overlaying interface in accordance with another embodiment, the overlaying interface having an open distal end;

FIG. 7 is an axonometric view from the front and top of a kit comprising an inner member and an adjustable outer sheath in accordance with another embodiment of the present invention;

FIG. 8 is a schematic of a method for mounting a prosthesis to a residual limb of a user in accordance with another embodiment of the present invention;

FIG. 9 is a cross-sectional view of the adjustable overlaying interface as shown in FIG. 4C taken along cross-section lines 9-9 thereof;

It will be appreciated that for simplicity and clarity of illustration, elements shown in the figures have not necessarily been drawn to scale. For example, the dimensions of some of the elements may be exaggerated relative to other elements for clarity. Nevertheless, for disclosure purposes, it should be understood that the relative proportions of the various elements as shown in the figures are disclosed.

### DETAILED DESCRIPTION

It will be appreciated that, for simplicity and clarity of illustration, where considered appropriate, reference numerals may be repeated among the Figures to indicate corresponding or analogous elements or steps. In addition, numerous specific details are set forth in order to provide a thorough understanding of the exemplary embodiments described herein. However, it will be understood by those of ordinary skill in the art, that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way but rather as merely describing the embodiment of the various embodiments described herein. The embodiments, geometrical configurations, materials mentioned and/or dimensions shown in the figures are optional, and are given for exemplification purposes only.

Moreover, it will be appreciated that positional descriptions such as “above”, “below”, “forward”, “rearward”, “left”, “right” and the like should, unless otherwise indicated, be taken in the context of the figures only and should not be considered limiting. Moreover, the figures are meant to be illustrative of certain characteristics of the adjustable residual limb-prosthesis interface and are not necessarily to scale.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

In the following description, the expression “prosthesis socket” refers to a device forming at least partially a junction between a residual limb and a prosthesis. The prosthesis socket might be part of the socket or be mountable thereto.

In the following description, the expression “prosthetic liner” usually refers to a liner acting as an interface between the user's skin and their prosthesis. The prosthetic liner is thus engageable, either directly or indirectly, with the prosthesis socket and the residual limb of the user.

Other objects, advantages and features of the present description will become more apparent upon reading of the following non-restrictive description of specific embodiments thereof, given by way of example only with reference to the accompanying drawings.

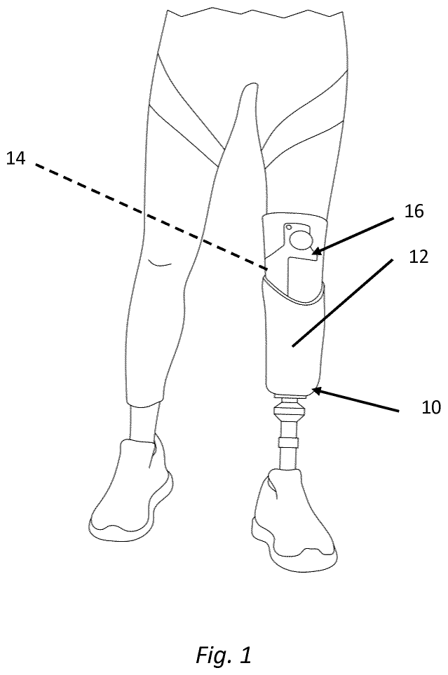

FIG. 1, now referred to, depicts a user wearing a prosthesis 10. The prosthesis 10 is provided with a socket 12, in which is inserted a user's residual limb 14. Between the residual limb 14 and the socket 12, the user wears an adjustable overlaying interface 16 (also referred to as an adjustable sheath or a liner-covering sheath) which is shown extending above the socket 12 (i.e., having a proximal portion extending beyond an upper edge of the socket 12).

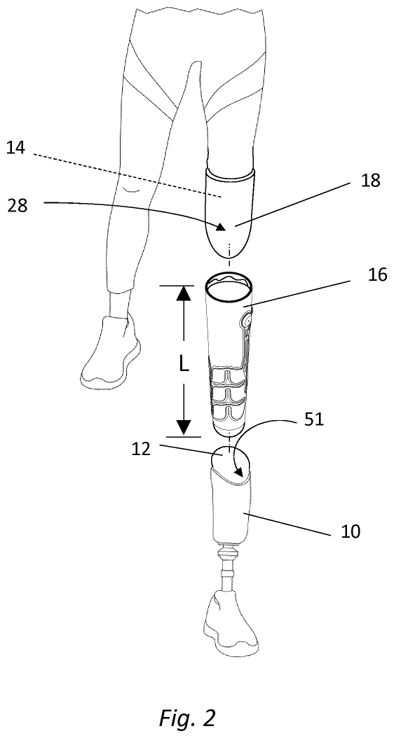

FIG. 2, now concurrently referred to, depicts how the different components are assembled together in order to work in the embodiment shown. First, the user must install a liner 18 on their residual limb 14, either directly or indirectly on the skin thereof. Such liners 18 are typically unrolled on the residual limb 14. Once the liner 18 has been donned on the residual limb 14, the adjustable overlaying interface 16 is installed by pulling it, and optionally stretching it, over the liner 18 (i.e., superposing it at least partially over an outer surface thereof). Then, the residual limb 14 donned with both the liner 18 and the adjustable overlaying interface 16 is inserted into the socket 12 of the prosthesis 10. Before describing how the prosthesis 10 is adjusted on the residual limb 14 using the adjustable overlaying interface 16, more details will be provided on the various components of the assembly forming an interface between the residual limb and the prosthesis socket.

Liners typically require to be unrolled on the residual limb 14 because they are designed to provide a high level of adhesion against the skin of the residual limb 14. Liners 18 achieve such high adhesion by using a combination of factors such as a pressure against the residual limb 14, which pressure may be dependent on an elasticity (defined for instance by the material's Young's modulus), thickness of the liner 18 and on a friction coefficient. Liners 18 generally have a high friction coefficient. The liners are usually at least partially made of silicone, polyurethanes, TPE and the like, which are known to be materials with a high friction coefficient. For example, the friction coefficient between silicone and skin ranges between 1.0 and 3.0, the friction coefficient between polyurethanes and skin ranges from 0.5 to 0.7 while the friction coefficient between TPE and skin ranges between 0.8 and 3.0. Moreover, the use of a relatively large wall thickness of the liners typically ranging from 3 mm to more than 9 mm and a relatively high Young's modulus (silicones liners typically ranging from 150 kPa to 3500 kPa, polyurethane liners typically ranging from 100 kPa to 250 kPa, and TPE liners typically ranging from 100 kPa to 2500 kPa) all contribute to achieve this desired high level of adhesion between the liner 18 and the residual limb 14. Liners are used traditionally to provide cushioning to the residual limb by distributing pressure on the limb caused by a prosthetic device. The liner also protects the limb from irritation that might be caused by movement of the prosthesis against the limb. Since such liners 18 are well known in art, they will not be further described.

The adjustable overlaying interface 16 (or prosthetic liner-recovering sheath) is used to more precisely adjust the prosthesis 10 to the liner 18 worn over the residual limb 14 of the user. More precisely, the adjustable overlaying interface is designed to compensate a variation of volume of the residual limb 14. This variation of volume occurs especially where there is muscular mass. For example, on a leg, the calf region would be more prone to volume variation than the front of the leg. The adjustable overlaying interface 16 may be shaped and dimensioned to be engaged with an amputated leg or an amputated arm. The leg amputation may be at any location of the leg, such as a knee disarticulation, tibial or femoral amputation. Similarly, the arm amputation could be, without being limitative, a transhumeral or transradial amputation, a wrist disarticulation and the like.

The adjustable overlaying interface 16 comprises a sleeve 20 (also referred to as a sheath body), an inflatable bladder 22 (also referred to as a variable-volume pad) and a fluid-providing device 24 fluidly connected to the inflatable bladder 22 for pressurizing, or pumping, a fluid into it and thereby inflate the inflatable bladder 22 (i.e., increase a volume thereof). As detailed below, the inflatable bladder and the fluid-providing device at least partially form together a thickness-adjusting system which is mounted to or formed integral with the sleeve 20. The sleeve 20 is designed to be pulled on and/or superposed onto an outer surface of the liner 18 and is thereby adapted to be worn over the liner 18 and at least partially inside the socket 12 of the prosthesis 10. For instance, the sleeve (or sheath body) is substantially tubular along at least a longitudinal portion thereof. The tubular sheath body (or sleeve) has an inner surface defining a cavity which is adapted to receive the liner 18. The variations in volume of the residual limb 14 may therefore be compensated by using the fluid-providing device 24 to inflate and/or deflate the inflatable bladder 22. If the fluid-providing device 24 is not of a model capable of pumping the fluid out of the inflatable bladder 22, the adjustable overlaying interface 16 may be further equipped with a valve 25, which is also fluidly connected to the inflatable bladder 22 in order to selectively let the fluid out of the inflatable bladder 22, in order to decrease the volume thereof.

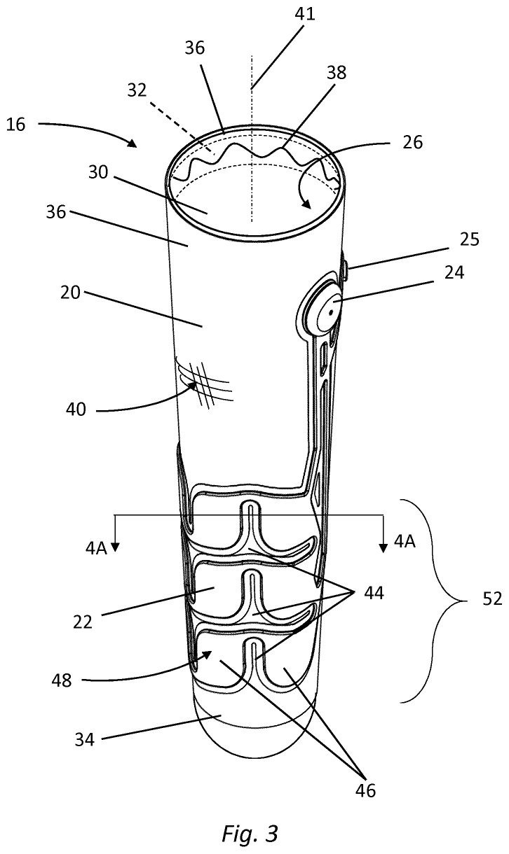

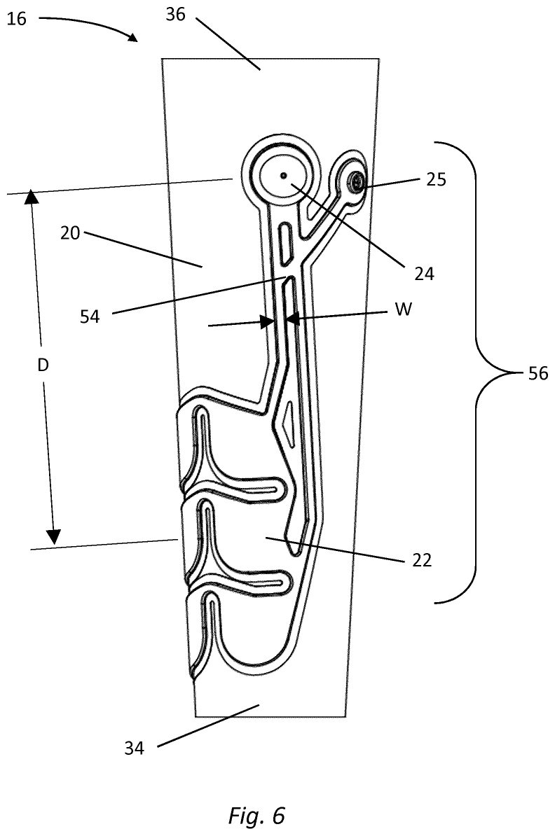

In the embodiment shown, the sleeve 20 is basically a tubular chassis—or comprises at least a substantially tubular portion—to which the inflatable bladder 22 is attached. The sleeve 20 depicted in FIG. 3 is shown with a closed distal end portion 34. However, this distal end portion 34 could also be open, as best shown in FIG. 6. Alternatively, the inflatable bladder 22 may be integrally formed with the sleeve 20. The sleeve 20 is made, at least partially, of a stretchable material so as to snugly fit over the liner 18 once pulled over it. For example, the sleeve 20 or its material may have a stretchability of at least 10% (for instance considered in a longitudinal direction thereof, i.e., considered along a longitudinal axis of the residual limb wearing the liner and the prosthetic liner-recovering sheath) and may have a Young's modulus of less than substantially 100 kPa. Moreover, at least a portion of an inner surface 26 of the sleeve 20 has a friction coefficient against human skin of less than approximately 1, even less than about 0.5 or less than about 0.4. This combination makes it convenient for the user to easily pull the adjustable overlaying interface 16 over an outer surface 28 of the liner 18. The stretchability of the sleeve 20 allows it to snuggly fit over the liner 18 without making wrinkles, while the relatively low Young's modulus and relatively low friction coefficient, which is in particular smaller than the friction coefficient of an inner surface of the liner 18, contribute to making it easy for the user to slide the adjustable overlaying interface 16 over the liner 18.

The inner surface 26 of the sleeve 20 may be provided with at least two distinct zones having two different friction coefficients. For example, a first zone 30, typically representing at least about 30%, for instance at least about 45%, for instance at least about 60% of a total inner surface area of the sleeve 20 may have a friction coefficient against human skin of less than 0.5 while the second zone 32 may have a friction coefficient against human skin greater than the friction coefficient of the first zone 30 yet still less than 1. The second zone 32 may be arranged at at least one of the distal end portion 34 and a proximal end portion 36 of the sleeve 20. The second zone 32 can, for example, be defined by a zone of the sleeve inner surface 26 which is provided with a material increasing the friction coefficient, or stickiness, such as a strip 38 of silicone or of a rubberized material.

Suitable fabrics may be used for manufacturing the sleeve 20. Conveniently, the sleeve material may also have antibacterial properties. By way of example, the following materials may be used for the sleeve 20: nylon, polyester, elastane, aramid fibers, cotton, wool, silk, thermoplastic polyurethane, polyurethane, polyvinyl chloride, natural fibers, or any combination thereof (such as for instance and without being limitative a micro-fiber base with a PU film lamination, knitted poly-spandex with PU film lamination or PU coating and the like). This list is not exhaustive as other materials may also be suitable. The material might be woven or non-woven.

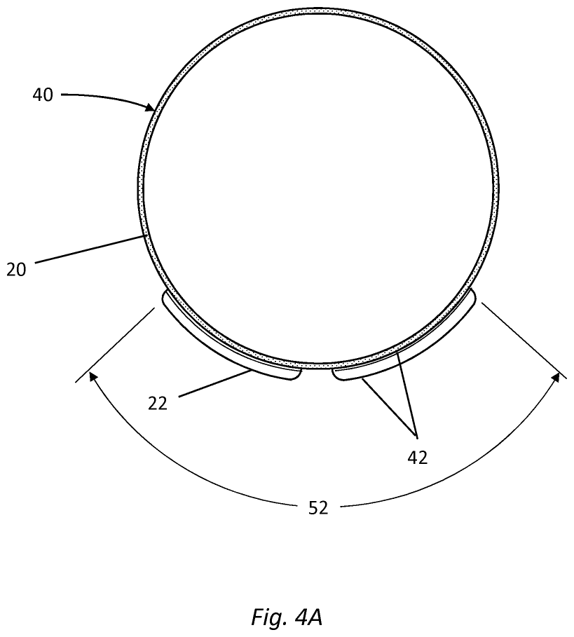

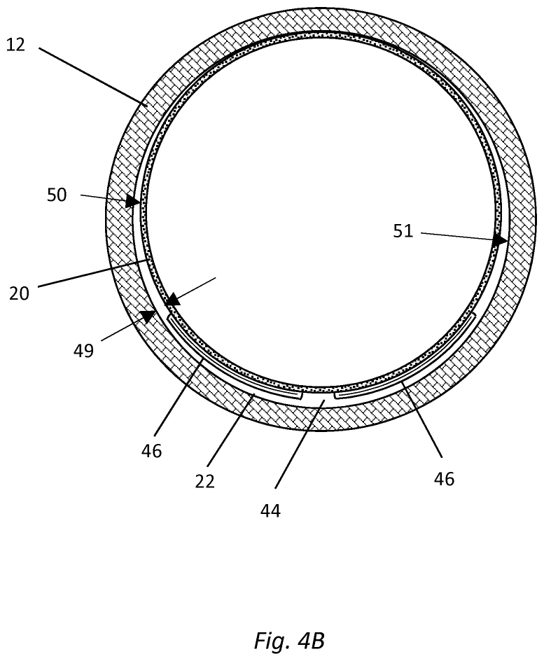

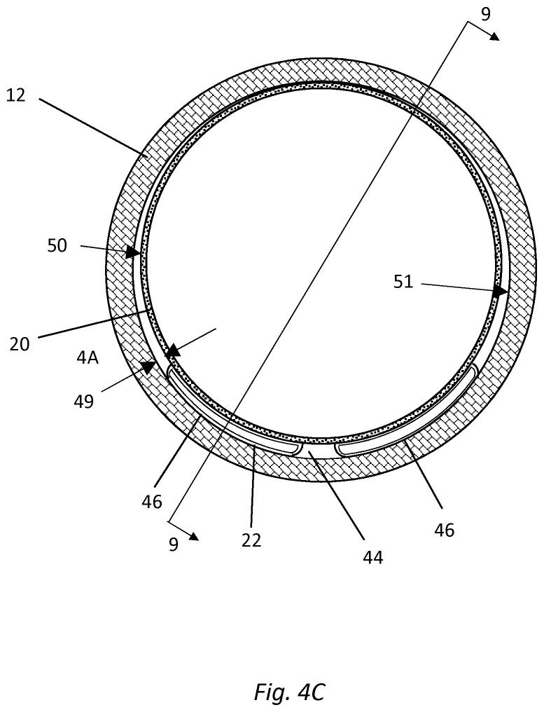

For instance, the inflatable bladder 22 is attached to the sleeve 20. It may be permanently attached, thereby providing a thin assembly of the adjustable overlaying interface 16, or removably. The inflatable bladder 22 is secured to a sleeve outer surface 40 or within it. This sleeve outer surface 40 is adapted to at least partially face an inner surface 51 of the socket 12 and is preferably made of a wear resistant material, such as nylon or aramid fiber, or any other suitable material. FIGS. 4A, now concurrently referred to, represents a cross-section of the adjustable overlaying interface 16 taken normal to a longitudinal axis 41. As best shown in FIG. 4A, the inflatable bladder 22 typically extends over less than 270 degrees of a circumference of the sleeve 20. It can be even less, for example less than 180 degrees of the circumference of the sleeve 20, but yet typically more than 90 degrees of the circumference of the sleeve 20.

As best shown in FIG. 2, the inflatable bladder 22 extends substantially along at least 30% of a length L of the adjustable overlaying interface 16. In a specific example, the inflatable bladder 22 extends substantially along at least 50%, 70% or even up to substantially 100% of the length of the adjustable overlaying interface 16.

In the embodiment shown, the inflatable bladder 22 comprises two walls 42 (FIG. 4A) sealed together (for instance welded together) on a periphery thereof. In order to better control how the inflatable bladder 22 inflates, it is possible to locally attach the two walls 42 within their periphery. For example, this local attachment of both walls 42 can be achieved by a weld line, a spot weld or an area weld of both walls 42 together within the periphery, but always ensuring that inflatable cells 46 thereby defined always remain in fluid communication with each other. Locally attaching both walls 42 thereby defines relief zones 44 where the inflatable bladder 22 cannot be inflated (i.e., where an inner volume of the inflatable bladder remains locally substantially constant or null) whereas inflatable cells 46 where the inflatable bladder 22 can be inflated (i.e., where the inner volume of the inflatable bladder is variable). All cells 46 are fluidly interconnected so that the fluid with which the inflatable bladder 22 is inflated can circulate from one cell 46 to another cell 46 and inflate all cells 46 at once. It could however be envisioned to have an adjustable overlaying interface 16 which would comprise a plurality of inflatable bladders which are independently inflatable. The inflatable cells 46 create a visible pattern 48 on an outer surface 50 of the adjustable overlaying interface 16. Conveniently, the pattern 48 provides an indication of where an adjustable zone 52 is located so that the user may align the adjustable overlaying interface 16 with a predetermined muscular portion of the residual limb 14 which is typically more prone to varying in volume. Although the size and shape of the inflatable cells 46 may be experimented with, it has been found that inflatable cells 46 fitting within a circle of about 100 mm of diameter, for instance fitting within a circle of about 60 mm of diameter, for instance fitting within a circle of about 40 mm of diameter, for instance fitting within a circle of about 20 mm of diameter work well. The pattern 48 extends along the longitudinal axis of the sleeve 41. It shall be noted that the cells shape may have various shape and the fact that they are described as fitting within a circle do not mean that they must be round in shape. Cells 46 could also be defined differently, for example, by their surface area or volume. For instance, at least one of the inflatable cells 46 has an area smaller than about 8000 mm², for instance smaller than about 6000 mm², for instance smaller than about 3000 mm², for instance smaller than about 1000 mm².

FIG. 4B depicts the same cross-section of the same adjustable overlaying interface 16 as in FIG. 4A, except that a cross-section of an installed socket 12 of a prosthesis 10 has been added to better illustrate a gap 49 or space that the inflatable bladder 22 is intended to at least partially and selectively compensate for. The relief zone 44 is also illustrated in between two inflatable cells 46. As the residual limb 14, donned with the liner 18 would vary in volume, the sleeve 20 would still closely fit the liner 18 because of the elasticity of the sleeve 20, but the gap 49 would vary in size since the socket 12 is rigid. As can be seen in FIG. 4B, when the residual limb 14 decreases in volume, the gap 49 appears between the sleeve 20 and the inner surface of the socket 12, which leads to a discomfort for the user. FIG. 4B depicts the inflatable bladders 22 in an underinflated state, or even completely deflated. When such discomfort occurs because the residual limb 14 decreased in volume, the user may either starts inflating, or further inflates the inflatable bladders 22 using the fluid-providing device 24. The inflated state of the inflatable bladders is depicted in FIG. 4C, now concurrently referred to. Conversely, the user may use the valve 25 to decrease the pressure within the inflatable bladders 22 if the volume of their residual limb 14 increases and discomfort is felt because of retrained volume within the socket 12. As can be seen, the gap 49 is not constant all around the sleeve 20, a consequence of the inflatable bladder 22 not extending all around the adjustable overlaying interface 16, but rather being concentrated on a zone where the residual limb 14 is more prone to vary in volume.

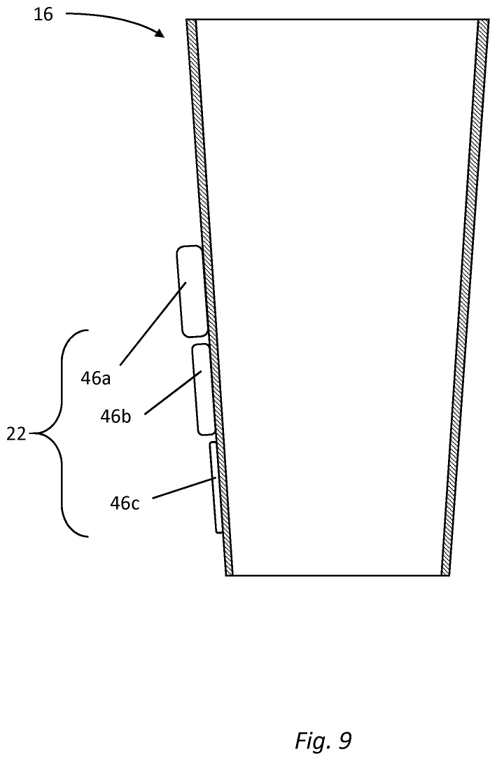

By at least partially inflating the inflatable bladder 22, the adjustable overlaying interface 16 is at least partially expanded in a direction substantially transversal to the longitudinal axis 41 (i.e., substantially radially in an outward direction, when the overlaying interface has a substantially cylindrical shape when in use and with reference to a liner-receiving cavity at least partially delimited by the inner surface of the overlaying interface), and the outer surface 50 of the adjustable overlaying interface 16, or at least portions thereof formed by the inflatable bladder 22, is displaced towards the inner surface 51 of the socket 12. Conversely, by at least partially deflating the inflatable bladder 22, the adjustable overlaying interface 16 is at least partially compacted in a direction substantially transversal to the longitudinal axis 41 (i.e., substantially radially in an inward direction, when the overlaying interface has a substantially cylindrical shape when in use and with reference to the liner-receiving cavity at least partially delimited by the inner surface of the overlaying interface), and the outer surface 50 of the adjustable overlaying interface 16, or at least the portions thereof formed by the inflatable bladder 22, are displaced away from the inner surface 51 of the socket 12. For instance, the inflatable bladder is shaped and dimensioned to have an inflated thickness greater than about 5 mm, for instance greater than about 10 mm, for instance greater that about 20 mm, for instance greater than about 30 mm, for instance greater than about 40 mm. The inflatable bladder might be shaped and dimensioned so that at least some of the inflatable cells thereof have different inflated thicknesses, in order to increase an adjustment of the prosthetic liner-recovering sheath to the morphology of the user. This is illustrated in FIG. 9 where the inflatable cells 46 are increased to different extent because of their configuration. For example, small cell 46 could be designed to inflate to a lesser extent than larger cells 46. This is depicted in FIG. 9, now concurrently referred to, where cells 46a, 46b and 46c are all inflated to different extent. Indeed, it can be seen that cell 46a is extended to a thickness which is more than both cell 46b, which itself is extended to a thickness which is more than cell 46c. This is based on their physical configuration since the pressure is the same within all cells 46, as they are interconnected. This variation in the thickness of different cells 46 may be beneficial in order to better adapt to various requirements of different regions of the residual limb 14, or simply to provide a better comfort to one user which may feel differently from another user for the same region of the residual limb 14.

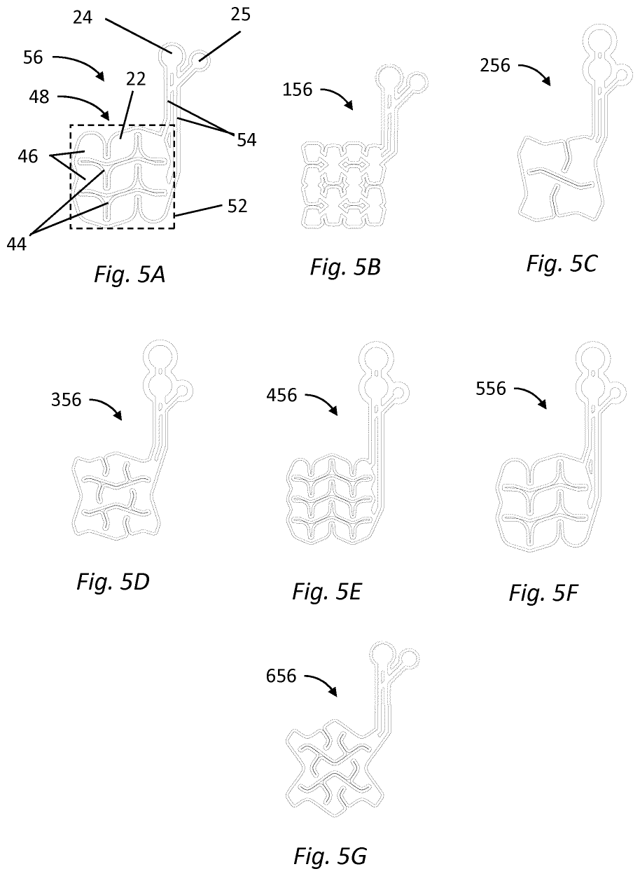

FIGS. 5A to 5G, now concurrently referred to, schematically represents different possible embodiments of thickness-adjusting systems 56, 156, 256, 356, 456, 556 and 656 each having cell patterns and/or different relative arrangements of fluid-providing devices and/or valves. As with the shape of the inflatable cells 46, the shape of the adjustable zone 52 may be experimented with in order to suit a morphology of a user. It has however been found that square or rectangular adjustable zones 52, the rectangular zone 52 extending in its longer dimension along the direction of the longitudinal axis 41, provide satisfying results. For instance, the pattern 48 can typically comprise between 1 and about 20 cells fluidly connected to each other. The cells shape can vary from one configuration to another. A single inflatable bladder 22 can even use different shapes of cells 46.

The inflatable bladder 22 may be made, at least partially, of either stretchable or non-stretchable materials that are impervious to the fluid being used. The inflatable bladder 22 may be constructed of a thin material such as a film, typically made of thermoplastic polyurethane, thermoplastic elastomer, polyurethane, silicone, rubber or polyvinyl chloride or a combination thereof. This is not an exhaustive list and other suitable materials may be used. Conveniently, especially if the inflatable bladder 22 is made of a stretchable material, the sleeve 20 and the inflatable bladder 22 may be made of the same material such that the sleeve 20 constitutes one of the walls 42 of the inflatable bladder 22. In any case, it is preferable to select materials so that the thickness of the adjustable overlaying interface 16 along substantially an entirety of the periphery thereof remains less than 3 mm, or even less than 2 mm, when the inflatable bladder 22 is fully deflated. It may even be less than that, for example, less than 1.5 mm.

According to some example embodiments, the inflatable bladder 22 may be aligned with a muscle and/or bone of the residual limb for increased blood flow and comfort. For example, the inflatable bladder 22 may be aligned, at least partially, with a triceps sural, tibialis anterior, fibularis longus, extensor hallucis longus, medial gastrocnemius, lateral gastrocnemius, soleus, flexor hallucis longus, fibularis brevis (peroneus brevis), biceps femoris, and/or plantaris muscle(s) of the user. Furthermore, the inflatable bladder 22 may be aligned with a back of the leg, over an area that may extend from the intercondylar eminence or popliteal fossa to the calcaneal (Achilles) tendon, the calcaneus, or to the medial and lateral malleolus.

FIG. 6 is now referred to. The fluid-providing device 24 is adapted to pump the fluid which is intended to be used. Preferably, a compressible fluid such as air or CO₂ may be used. Alternatively, an incompressible fluid such as a liquid like water could be used. This is however likely a less practical option as, for example, a storage reservoir would be required. Nevertheless, in case of the use of an incompressible fluid, the fluid-providing device 24 needs to be adapted to pump such incompressible fluid and the inflatable bladder 22 would become an expandable bladder and the inflatable cells 46 would become expandable cells. In such a case, the incompressible Air is convenient as it is readily available from the environment. Both the fluid-providing device 24 and the valve 25 are located proximate the proximal end portion 36 of the sleeve 20 so as to be outside the socket 12 when in use, as is best shown in FIG. 1, and easily reachable by the user. The fluid-providing device 24 is fluidly connected to the inflatable bladder 22. As best shown in FIG. 6, the fluid-providing device 24 and the valve 25 may be longitudinally spaced apart from the inflatable bladder 22, considered relative to the longitudinal direction of the overlaying interface, and are then connected by one or more conduit 54. For instance, the conduit 54 has a width W, considered in a direction transversal to the longitudinal direction of the prosthetic liner-recovering sheath, smaller than about 20 mm, for instance smaller than about 15 mm, for instance smaller than about 10 mm, for instance smaller than about 5 mm, for instance of about 1 mm. For instance, a distance D between at least one of the fluid-providing device 24 and the valve 25 and the inflatable bladder 22 is greater than about 1 cm, for instance greater than about 3 cm, for instance greater than about 5 cm, for instance greater than about 10 cm. In all cases, the fluid-providing devices 24 and the valve 25 are connected to the cells 46 through at least one conduit 54. There may be one or more conduits 54 if it is deemed beneficial to do so, for example in order to reach simultaneously two inflatable bladders 22 disposed in two different areas of the adjustable overlaying interface 16 (for example, the distal end portion 34 and the proximal end portion 36), or to reach two areas of the same pattern 48. This may be beneficial when two inflatable bladders 22 need to be inflated simultaneously in order not to create an excessive pressure in the area where the first bladder is located if only the second bladder is inflated.

The fluid-providing device 24 is typically one of a pump, either manual or electric, and a pressurized cartridge of compressed fluid, for example air or CO₂. If used, the cartridge could be stored in a storage pouch affixed to the sleeve 20. In the present FIGS. 1 to 6, the fluid-providing device 24 has been represented as a manual pump, but that should not be considered as limiting. The fluid-providing device 24 may also be a battery-powered pump which could even be automated when used with pressure sensors sensing the pressure within the inflatable bladder 22 or on the sleeve 20 adjacent the inflatable bladder 22. For example, the adjustable overlaying interface 16 could further comprise one or more pressure gauges, regulators and pressure sensors cooperating with the fluid-providing device 24 for adjusting the pressure inside the inflatable air bladder 22.

An assembly of the fluid-providing device 24 and the inflatable bladder 22 constitutes a thickness-adjusting system 56. Optionally, the thickness-adjusting system 56 may include the valve 25. The thickness-adjusting system 56 is best shown in FIG. 6.

For instance, the thickness-adjusting system is permanently secured to the sheath body. Unless otherwise stated, the term permanent implies that the thickness-adjusting system can not be separated from the sheath body without at least partially damaging at least one of the thickness-adjusting system and the sheath body. For instance, the thickness-adjusting system is secured to the sheath body (for instance to an outer surface thereof) via a permanent adhesive layer, High Frequency welding (HF Welding), Radio-frequency welding (RF Welding), heat press bonding, heat welding, ultrasonic welding, or could be arranged with a closed pocket at least partially formed by the sheath body. The thickness-adjusting system could also be arranged between two layers or two components at least partially forming the sheath body. The thickness-adjusting system could also be formed at least partially integral with the sheath body: for instance, it could be conceived that the sheath body be formed at least partially of a material impervious to the fluid (for instance at least partially formed of poly-spandex with a laminated TPU layer forming one of the walls of the thickness-adjusting system, the second wall of the thickness-adjusting system being welded and/or glued to the sheath body). In another embodiment, the thickness-adjusting system is removably mounted to the sheath body, for instance removably engageable in a pocked or bladder-receiving cavity formed in the sheath body.

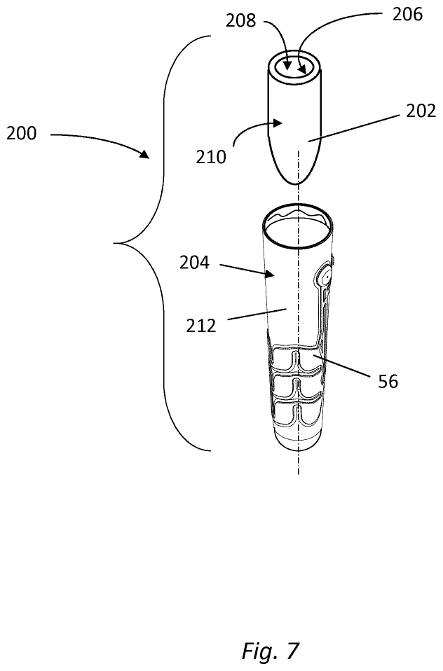

FIG. 7 is now concurrently referred to. In accordance with another embodiment of the present invention, a kit 200 for adjusting a prosthesis to a residual limb of a user is provided. The kit 200 comprises an inner member 202, substantially corresponding to the liner 18, and an adjustable outer sheath 204, substantially corresponding to the adjustable overlaying interface 16 or prosthetic liner-recovering sheath. The inner member 202 has a limb-facing surface 206 at least partially delimiting a limb-receiving cavity 208 and being superposable onto the residual limb 14 of the user. The inner member 202 also has an outer surface 210 opposed the limb-facing surface 206. The adjustable outer sheath 204 comprises a sheath body 212, substantially corresponding to the sleeve 20, and a thickness-adjusting system 56. The sheath body 212 is adapted to at least partially overlay the outer surface 210 of the inner member 202. The thickness-adjusting system 56 is secured to or is formed integral with the sheath body 212. The prosthetic liner-recovering sheath might be at least partially secured to the outer surface of the inner member, for instance be sewed, glued or welded thereto, or be removably mounted thereto (for instance via a pocket formed in the liner, or any suitable removable fasteners).

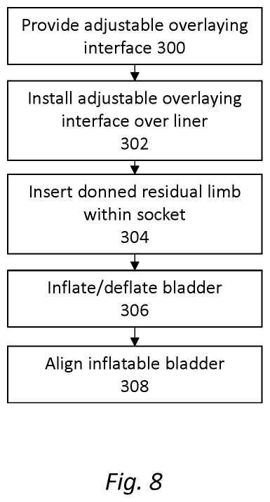

FIG. 8 is now concurrently referred to. In accordance with another embodiment of the present invention, a method for mounting a prosthesis to a residual limb of a user is provided. The method comprises:
a) providing 300 the adjustable overlaying interface 16;
b) installing 302 the adjustable overlaying interface 16 over the liner 18 (302) donned over the residual limb 14;
c) at least partially inserting 304 the residual limb 14 wearing the liner 18 and the adjustable overlaying interface 16 within the socket 12 of the prosthesis 10; and
d) either inflating or deflating 306 the inflatable bladder 22 of the adjustable overlay interface 16 to compensate variations in volume of the residual limb 14.

In more details, the action of installing 302 the adjustable overlaying interface further comprises pulling the sleeve 20 of the adjustable overlaying interface 16 over the residual limb 14. Also, the user may find it beneficial to e) align 308 the inflatable bladder 22 of the adjustable overlaying interface 16 over a predetermined muscular portion of the residual limb 14, which is typically more prone to varying in volume. In particular, and if the adjustable overlaying interface is so equipped, the user may activate the pump of the fluid-providing device 24 to do the inflating 306. Such inflating 306 can be inflating to a predetermined pressure inside the inflatable bladder 22. If the user has exceeded the predetermined pressure, or if the user overinflated the inflatable bladder 22 to a point where it is not comfortable, the user may deflate the inflatable bladder 22 to the predetermined pressure or below using the valve 25.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind. The scope of the invention is therefore intended to be limited by the scope of the appended claims.

1. An adjustable overlaying interface for adjusting a prosthesis to a liner worn over a residual limb of a user, the adjustable overlaying interface comprising:
a sleeve, the sleeve being pullable on an outer surface of the liner, the sleeve being thereby adapted to be worn over the liner and at least partially inside a socket of the prosthesis;
an inflatable bladder, the inflatable bladder being attached to or integrally formed with the sleeve; and
a fluid-providing device, the fluid-providing device being fluidly connected to the inflatable bladder, the fluid-providing device being operative to inflate the inflatable bladder with a fluid;
wherein variations in volume of the residual limb are compensatable by using the fluid-providing device to inflate and/or deflate the inflatable bladder.

2. The adjustable overlaying interface of claim 1, further comprising a valve, the valve being fluidly connected to the inflatable bladder to selectively let the fluid out of the inflatable bladder.

3. The adjustable overlaying interface of claim 2, wherein both the fluid-providing device and the valve are located proximate a longitudinal extremity of the sleeve so as to be outside the socket when in use.

4. The adjustable overlaying interface of claim 1, wherein the sleeve is at least partially made of a stretchable sleeve material so as to snugly fit over the liner.

5.-7. (canceled)

8. The adjustable overlaying interface of claim 1, wherein the sleeve is at least partially made of a material selected from the list consisting of: nylon, polyester, elastane, aramid fibers, cotton, wool, silk, thermoplastic polyurethane, polyurethane, polyvinyl chloride or a combination thereof.

9. (canceled)

10. The adjustable overlaying interface of claim 1, wherein at least a portion of an inner surface of the sleeve has a sleeve friction coefficient against human skin of less than substantially 1.

11. The adjustable overlaying interface of claim 10, wherein the inner surface of the sleeve has a first zone, the first zone having a friction coefficient against human skin of less than 0.5.

12. The adjustable overlaying interface of claim 11, wherein the inner surface of the sleeve further comprises a second zone, the second zone having a friction coefficient against human skin greater than the friction coefficient of the first zone and less than 1.

13. The adjustable overlaying interface of claim 12, wherein the second zone is arranged at at least one of distal and proximal end portions of the sleeve.

14. (canceled)

15. The adjustable overlaying interface of claim 1, wherein the inflatable bladder is secured to an outer surface of the sleeve, at least a portion of the outer surface of the sleeve being adapted to face an inner surface of the socket.

16.-18. (canceled)

19. The adjustable overlaying interface of claim 1, wherein the inflatable bladder comprises two walls sealed together on a periphery thereof, the two walls being further attached together locally and within their periphery so as to define relief zones where the inflatable bladder is uninflatable and a plurality of inflatable cells where the inflatable bladder is inflatable, the outer surface being adapted to at least partially face the socket.

20.-23. (canceled)

24. The adjustable overlaying interface of claim 1, wherein the inflatable bladder comprises a film made of a bladder material impervious to the fluid, the bladder material being selected from the list consisting of: thermoplastic polyurethane, thermoplastic elastomer, polyurethane, polyvinyl chloride or a combination thereof.

25. The adjustable overlaying interface of claim 1, wherein a thickness of the adjustable overlaying interface along substantially an entirety of a periphery thereof is less than 3 mm when the inflatable bladder is fully deflated.

26. (canceled)

27. The adjustable overlaying interface of claim 1, wherein the fluid-providing device is longitudinally spaced apart from the inflatable bladder.

28. The adjustable overlaying interface of claim 1, wherein the fluid-providing device is one of a pump and a cartridge of pressurized compressible fluid.

29. A prosthetic liner-recovering sheath for use with a prosthetic socket and an associated liner, the prosthetic liner-recovering sheath comprising:
a sheath body being substantially tubular along at least a longitudinal portion thereof and having a liner-superposing inner surface at least partially defining a liner-receiving cavity; and
a thickness-adjusting system secured to or formed integral with the sheath body and comprising at least one variable-volume pad arranged to be pressurized so as to adjust a thickness of the sheath along at least a portion of a periphery thereof.

30. The prosthetic liner-recovering sheath according to claim 29, wherein the thickness-adjusting system further comprises a fluid-providing device in fluid communication with the at least one variable-volume pad to respectively inflate and deflate the at least one variable-volume pad.

31.-57. (canceled)

58. A kit for adjusting a prosthesis to a residual limb of a user, the kit comprising:
an inner member having a limb-facing surface at least partially delimiting a limb-receiving cavity and being superposable onto the residual limb of the user, and an opposed outer surface, and
a prosthetic liner-recovering sheath according to claim 29 overlaying at least partially the outer surface of the inner member.

59. (canceled)

60. A method for mounting a prosthesis to a residual limb of a user, the method comprising:
providing an adjustable overlaying interface having:
a sleeve, the sleeve being pullable on an outer surface of the liner, the sleeve being thereby adapted to be worn over the liner and at least partially inside a socket of the prosthesis;
an inflatable bladder, the inflatable bladder being attached to or integrally formed with the sleeve; and a fluid-providing device, the fluid-providing device being fluidly connected to the inflatable bladder, the fluid-providing device being operative to inflate the inflatable bladder with a fluid;
wherein variations in volume of the residual limb are compensatable by using the fluid-providing device to inflate and/or deflate the inflatable bladder;
installing the adjustable overlaying interface over a liner donned over the residual limb of the user;
at least partially inserting the residual limb wearing the liner and the adjustable overlaying interface within a socket of the prosthesis; and
at least one of inflating and deflating the inflatable bladder of the adjustable overlay interface to compensate variations in volume of the residual limb.

61. The method of claim 60, wherein installing the adjustable overlaying interface comprises pulling the sleeve of the adjustable overlaying interface over the residual limb.

62.-65. (canceled)
